(19) United States
(12) Patent Application Publication
(10) Pub. No.: US 2019/0083053 A1
(43) Pub. Date: Mar. 21, 2019

(54) ENERGY SEPARATION IN MULTI-ENERGY COMPUTED TOMOGRAPHY

(71) Applicant: General Electric Company, Schenectady, NY (US)

(72) Inventor: Brian Douglas Lounsberry, Thiensville, WI (US)

(21) Appl. No.: 15/711,939

(22) Filed: Sep. 21, 2017

(51) Int. Cl. A61B 6/00; G01N 23/04; A61B 6/03

(52) U.S. Cl. CPC A61B 6/5205; G01N 23/046; A61B 6/40; A61B 6/4208; A61B 6/4452; A61B 6/032

(57) ABSTRACT

In accordance with the present approach, a kV switched X-ray source, such as a kV switched X-ray tube, is used in conjunction with a dual-layer detector. In such approaches, the dual-layer detector may be operated so as to ignore or discard signal attributable to low-energy photons generated during a high kV emission interval or view.

### BACKGROUND

The subject matter disclosed herein relates to multi-energy X-ray imaging.

Non-invasive imaging technologies allow images of the internal structures or features of a patient to be obtained without performing an invasive procedure on the patient. In particular, such non-invasive imaging technologies rely on various physical principles, such as the differential transmission of X-rays through the target volume or the reflection of acoustic waves, to acquire data and to construct images or otherwise represent the observed internal features of the patient.

For example, in computed tomography (CT) and other X-ray-based imaging technologies, X-ray radiation spans a subject of interest, such as a human patient, and a portion of the radiation impacts a detector where the intensity data is collected. In scintillator-based detector systems, a scintillator material generates optical or other low-energy photons when exposed to the X-ray and a photodetector then produces signals representative of the amount or intensity of radiation observed on that portion of the detector. The signals may then be processed to generate an image that may be displayed for review. In CT systems, this X-ray transmission information is collected at various angular positions as a gantry is rotated around a patient to allow volumetric reconstructions to be generated.

In clinical practice it may be desirable to acquire such X-ray transmission data at more than one X-ray energy, or spectrum, as the difference in X-ray transmission at the different energies can be leveraged to generate images corresponding to different tissue types or conveying information related to the spatial material composition within the imaged region. Such approaches, in a computed tomography context, may be characterized as spectral CT, dual-energy CT or multi-energy CT.

As discussed herein, the spectra may be characterized by the maximum operating voltage (kVp) of an X-ray tube used to generate the X-rays, also denoted as the operating voltage level of the X-ray tube. Though such X-ray emissions may be generally described or discussed herein as being at a particular energy level (e.g., referring to the electron beam energy level in a tube with an operating voltage of, for example, 70 kVp, 150 kVp, and so forth), the respective X-ray emissions actually comprise a continuum or spectrum of energies and may, therefore, constitute a polychromatic emission centered at, terminating at, or having a peak strength at, the target energy.

Such multi-energy imaging approaches necessitate being able to separate the signal attributable to different energy spectra or to different regions of a single spectrum, i.e., good energy separation. Current approaches to achieve energy separation all have drawbacks or tradeoffs related to poor separation of the different energy levels or poor synchronicity, i.e., a temporal offset between when corresponding signals for different spectra are acquired, and/or poor radial correspondence, i.e., the different energy signals may be acquired at radially offset positions from one another using separate emission and detection components. Temporal offset errors are the main drawback in dual tube systems and systems employing a single tube switched between emission states. Conversely, systems employing a dual-layer detector that generates two energy signals for a single spectrum emission suffer from relatively poor energy separation because there is overlap of the measured low- and high-energy spectra across the entire spectral range. With this in mind, improvements in energy separation in a spectral CT context may be useful.

### BRIEF DESCRIPTION

In one embodiment, a method of acquiring and processing dual-energy X-ray transmission data is provided. In accordance with this method, a first X-ray beam having a first keV distribution and a second X-ray beam having a second keV distribution different than the first keV distribution are alternately emitted from an X-ray source. In response to each emitted first X-ray beam, at least a low-energy scintillator signal is read out from a first layer of a dual-layer detector. In response to each emitted second X-ray beam, at least a high-energy scintillator signal is read out from a second layer of the dual layer detector. At least the low-energy scintillator signals and the high-energy scintillator signals are processed to generate an image.

In a further embodiment, an imaging system is provided. In accordance with this embodiment, the imaging system includes: an X-ray source configured to be switched during operation between a first operating voltage corresponding to a first emission spectrum and a second operating voltage corresponding to a second emission spectrum; a dual-layer X-ray detector having a first layer and a second layer; a data acquisition system configured to read out at least the first layer when the X-ray source is operated at the first operating voltage and to read out at least the second layer when the X-ray source is operated at the second operating voltage; and image processing circuitry configured to generate an image using signals acquired from at least the first layer when the X-ray source is operated at the first operating voltage and using signals acquired from only the second layer when the X-ray source is operated at the second operating voltage.

In an additional embodiment, a method is provided for acquiring dual-energy X-ray data. In accordance with this method at least a low-energy scintillator layer of a dual-layer detector is read out to generate first signals when the dual-energy detector is irradiated by an X-ray source operated at a first operating voltage. A high-energy scintillator layer of the dual-layer detector is read out to generate second signals when the dual-energy detector is irradiated by the X-ray source operated at a second operating voltage. A tissue-type or material decomposition image is generated using the first signals and the second signals.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a schematic illustration of an embodiment of a computed tomography (CT) system configured to acquire CT images of a patient and process the images in accordance with aspects of the present disclosure;

FIG. 2 depicts a generalized representation of features of a dual-layer detector, in accordance with aspects of the present disclosure;

FIG. 3 depicts examples of low- and high-energy spectra observed using a dual-layer detector alone;

FIG. 4 depicts examples of low- and high-energy spectra observed using a kV switched X-ray source alone;

FIG. 5 depicts diagrammatically use of a kV switched source and dual-layer detector in accordance with aspects of the present disclosure; and

FIG. 6 depicts examples of low- and high-energy spectra observed using both a kV switched X-ray source and dual-layer detector.

### DETAILED DESCRIPTION

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

While the following discussion is generally provided in the context of medical imaging, it should be appreciated that the present techniques are not limited to such medical contexts. Indeed, the provision of examples and explanations in such a medical context is only to facilitate explanation by providing instances of real-world implementations and applications. However, the present approaches may also be utilized in other contexts, such as the non-destructive inspection of manufactured parts or goods (i.e., quality control or quality review applications), and/or the non-invasive inspection of packages, boxes, luggage, and so forth (i.e., security or screening applications). In general, the present approaches may be desirable in any imaging or screening context in which dual- or multi-energy imaging is desirable, such a spectral computed tomography (CT).

Tissue characterization or classification may be desirable in various clinical contexts to assess the tissue being characterized for pathological conditions and/or to assess the tissue for the presence of various elements, chemicals or molecules of interest. Such approaches typically involve use of dual-energy imaging, i.e., data acquisition at a high-energy spectrum and a low-energy spectrum, i.e., two spectra having different mean keV's.

Such dual-energy imaging approaches typically take one of three forms: (1) use of a high-energy X-ray tube and detector and a separate, radially-offset, low-energy X-ray tube and detector (i.e., a dual tube/detector configuration); (2) use of a dual-layer detector where different layers of the detector, alone or in combination, are used to generate respective signals corresponding to low-energy X-ray photons and high-energy X-ray photons from a single emitted spectrum (i.e., a dual-layer detector configuration); or (3) use of an X-ray source that is rapidly switched between high- and low-energy X-ray emissions so as to allow different energy signals to be generated using a single X-ray tube and single-layer detector (i.e., a kV switching or fast-kV switching implementation). Each approach has its own advantages and disadvantages.

For example, dual tube/detector and kV switching methods can both be classified as ‘dual kV’ imaging techniques because both approaches use X-ray beams of different energies or mean keV's that are passed through the anatomy at different time intervals or azimuthal positions as the X-ray source(s) 16 and detector(s) are rotated around the object being imaged. Correspondingly, there is a temporal offset between when corresponding high- and low-energy signals are generated, with the temporal offset typically being greater in dual-tube approaches since the two tubes are radially offset from one another when rotated, in contrast to the single, switched tube in a kV switched approach. Conversely, the dual-layer detector approach uses a single X-ray beam (i.e., emitted spectrum) in a single time interval, and energy differentiation is performed within the two layers of the detector (i.e., for one X-ray emission spectrum, a high- and a low-energy detector signal are generated with no temporal offset between the signals). As a consequence, the dual-layer detector technique provides good temporal resolution but relatively poor energy separation because there is overlap of the low- and high-energy spectra across the entire keV range. Such overlap does not occur with the dual kV methodologies, where there is typically only overlap in the lower energy range.

Approaches based on kV switching offer an advantage over dual-tube/detector approaches in that the kV switching requires only a single X-ray source (that is toggled between high- and low-energy emitting modes) and a single detector. In contrast, dual-tube/detector approaches require two X-ray tubes and two detectors, increasing the cost and complexity of the system.

However, dual tube/detector methods offer an advantage over kV switching. In particular, since there are two, separate X-ray tubes creating the two different energy X-ray beams, the higher energy beam can be differentially filtered to move its mean energy higher, thereby reducing the overlap of energies in the low keV range. This provides greater energy separation than what is achievable with conventional kV-switching approaches. In particular, in order to perform comparable filtering in a single-tube kV switching scheme, a filter would need to be mechanically inserted into the beam during the high kV views (wherein a view is understood to be an exposure made at a particular rotational angle of the gantry) and then removed during the low kV views (obtained in alternation with the high kV views). Since these views are typically acquired in the kHz range, the mechanics of this solution are impractical.

With the preceding in mind, the present approach employs a kV switched X-ray source with a dual-layer detector to achieve improved energy separation without employing filters inserted into the beam path when high-kV views are acquired.

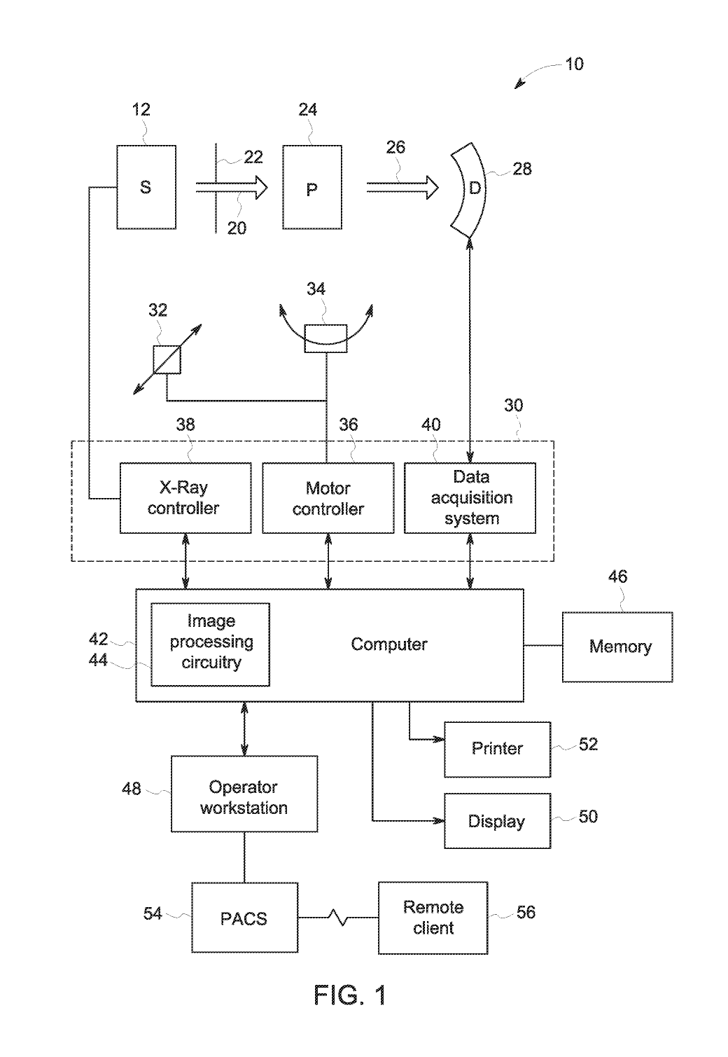

Prior to discussing the present approach for improving energy separation with kV switching, it may be useful to understand the operation and components of an imaging system that may be used to implement the present approach. With this in mind, FIG. 1 illustrates an embodiment of an imaging system 10 for acquiring dual-energy image data in accordance with aspects of the present disclosure. In the illustrated embodiment, system 10 is a computed tomography (CT) system designed to acquire X-ray projection data at multiple energy spectra, to reconstruct the projection data into volumetric reconstructions, and to process the image data, including material decomposition or tissue-type image data, for display and analysis. The CT imaging system 10 includes an X-ray source 12, such as an X-ray tube, which allows X-ray generation at multiple (e.g., two) spectra having different energy characteristics, during the course of an imaging session. For example, the emission spectra may differ in one or more of their mean, median, mode, maximum, or minimum X-ray energies.

By way of example, in one embodiment an X-ray source 12 (e.g., an X-ray tube) may be switched between a relatively low-energy polychromatic emission spectrum (e.g., X-ray tube operating voltage at about 80 kVp) and a relatively high-energy polychromatic emission spectrum (e.g., at about 140 kVp). As will be appreciated, the X-ray source 12 may emit at polychromatic spectra localized around energy levels (i.e., spectra induced by specific kVp ranges) other than those listed herein. Indeed, selection of the respective energy levels for emission may be based, at least in part, on the anatomy being imaged and the chemical or molecules of interest for tissue characterization.

In certain implementations, the source 12 may be positioned proximate to a beam shaper 22 used to define the size and shape of the one or more X-ray beams 20 that pass into a region in which a subject 24 (e.g., a patient) or object of interest is positioned. The subject 24 attenuates at least a portion of the X-rays. Resulting attenuated X-rays 26 impact a dual-layer detector array 28 formed by a plurality of detector elements (e.g., a one-dimensional or two-dimensional detector array) within each layer. Each detector element produces an electrical signal that represents the intensity of the X-ray beam incident at the position of the detector element when the beam strikes the detector 28. Electrical signals from the two layers of the detector 28 are acquired and processed to generate respective high- and low-energy scan datasets.

A system controller 30 commands operation of the imaging system 10 to execute examination protocols and to pre-process or process the acquired data. With respect to the X-ray source 12, the system controller 30 furnishes power, focal spot location, control signals and so forth, for the X-ray examination sequences. The detector 28 is coupled to the system controller 30, which commands acquisition of the signals generated by the detector 28. In addition, the system controller 30, via a motor controller 36, may control operation of a linear positioning subsystem 32 and/or a rotational subsystem 34 used to move components of the imaging system 10 and/or the subject 24.

The system controller 30 may include signal processing circuitry and associated memory circuitry. In such embodiments, the memory circuitry may store programs, routines, and/or encoded algorithms executed by the system controller 30 to operate the imaging system 10, including the X-ray source 12 and detector 28, such as to generate and/or acquire X-ray transmission data at two or more energy levels or bins, as well as to process the data acquired by the detector 28. In one embodiment, the system controller 30 may be implemented as all or part of a processor-based system such as a general-purpose or application-specific computer system.

The switched X-ray source 12 may be controlled by an X-ray controller 38 contained within the system controller 30. The X-ray controller 38 may be configured to provide power and timing signals to the source 12. As discussed herein, in certain implementations discussed herein, the X-ray controller 38 and/or the source 12 may be configured to provide fast-switching (i.e., near-instantaneous or view-to-view switching) of an X-ray source 12 between two (or more) energy levels. In this manner, the X-ray emissions may be rapidly switched between different kV's at which the source 12 is operated to emit X-rays at different respective polychromatic energy spectra in succession or alternation during an image acquisition session. For example, in a dual-energy imaging context, the X-ray controller 38 may operate an X-ray source 12 so that the X-ray source 12 successively (e.g., from view-to-view) emits X-rays at different polychromatic energy spectra of interest, such that adjacent projections are acquired at different energies (i.e., a first projection is acquired at low-energy, a second projection is acquired at high-energy, and so forth).

The system controller 30 may include a data acquisition system (DAS) 40. The DAS 40 receives data collected by readout electronics of the dual-layer detector 28, such as sampled digital or analog signals from the different layers of the detector 28. The DAS 40 may then convert the data to digital signals for subsequent processing by a processor-based system, such as a computer 42. In other embodiments, the detector 28 may convert the sampled analog signals to digital signals prior to transmission to the data acquisition system 40.

In the depicted example, the computer 42 may include or communicate with one or more non-transitory memory devices 46 that can store data processed by the computer 42, data to be processed by the computer 42, or instructions to be executed by a processor 44 of the computer 42. For example, a processor of the computer 42 may execute one or more sets of instructions stored on the memory 46, which may be a memory of the computer 42, a memory of the processor, firmware, or a similar instantiation. The memory 46 stores sets of instructions that, when executed by the processor 44, perform image acquisition and/or processing.

The computer 42 may also be adapted to control features enabled by the system controller 30 (i.e., scanning operations and data acquisition), such as in response to commands and scanning parameters provided by an operator via an operator workstation 48. The system 10 may also include a display 50 coupled to the operator workstation 48 that allows the operator to view relevant system data, imaging parameters, raw imaging data, reconstructed data, contrast agent density maps produced in accordance with the present disclosure, and so forth. Additionally, the system 10 may include a printer 52 coupled to the operator workstation 48 and configured to print any desired measurement results. The display 50 and the printer 52 may also be connected to the computer 42 directly or via the operator workstation 48. Further, the operator workstation 48 may include or be coupled to a picture archiving and communications system (PACS) 54. PACS 54 may be coupled to a remote client 56, radiology department information system (RIS), hospital information system (HIS) or to an internal or external network, so that others at different locations can gain access to the image data.

As noted above, the X-ray source 12 may be configured to emit X-rays at multiple energy spectra (e.g., dual-energy). Though such X-ray emissions may be generally described or discussed as being at a particular energy level (e.g., referring to the electron beam energy in a tube with an operating voltage typically in the range of about 70 kVp to about 150 kVp, the respective X-ray emissions actually comprise a continuum or spectrum of energies and may, therefore, constitute a polychromatic emission centered at, terminating at, or having a peak strength at, the target energy. For the purpose of material decomposition, such differing emission spectra allow attenuation data to be obtained for the same anatomical regions at the different spectra, thereby allowing differential attenuation at the different spectra to be determined for a given tissue or composition. Based on this differential attenuation at the known spectra, material and/or tissue decomposition techniques may be applied.

As discussed herein, in certain approaches an X-ray source 12 may be switched between low-energy and high-energy emitting states, with the resulting X-ray emission detected on a dual-layer detector 28 opposite the source 12 with respect to the imaged volume.

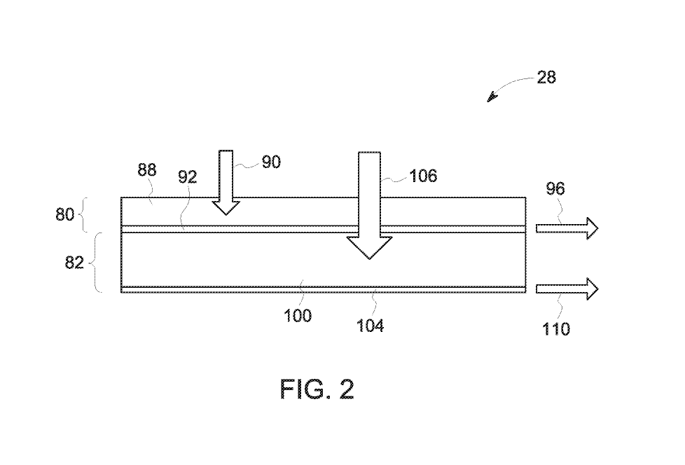

With the preceding in mind, and turning to FIG. 2 a generalized view of a dual-layer detector 28 is provided. In this depiction, the dual-layer detector 28 has a low-energy X-ray detection portion (depicted as low-energy detection layer 80) and a high-energy X-ray detection portion (depicted as high-energy detection layer 82). In the depicted example, the low-energy detection layer 80 is stacked above the high-energy detection layer 82 in the X-ray path such that X-rays encounter the low-energy detection layer 80 first, which in practice will act to stop those X-rays at a lower energy. Conversely, higher-energy X-rays that pass through the low-energy detection layer 80 go on to interact with the high-energy detection layer 82.

In the depicted example the low-energy detection layer 80 is formed of a low-energy scintillator 88 that has a depth and/or composition that interacts with lower energy X-rays 90 in the emitted spectra and corresponding low-energy readout circuitry 92. The interaction between the lower-energy X-rays 90 and the low energy scintillator 88 generates optical wavelength photons or other photons detectable by the low-energy signal readout circuitry 92 (e.g., photodiodes and associated readout circuitry) positioned to detect the photons generated by interaction of X-rays 90 and the low-energy scintillator 88. The readout circuitry 92 in turn generates electrical signals 96 indicative of the intensity of X-ray radiation interacting with the low-energy scintillator 88. As may be appreciated, the low-energy scintillator 88 may be subdivided in one- or two-dimensions by reflectors or septa so as to be a pixelated scintillator, with pixels of the scintillator 88 corresponding to readout elements of the readout circuitry 92. For the purpose of nomenclature and to facilitate discussion herein, the electrical signals 96 generated by X-ray interactions with the low-energy scintillator are referred to as low-energy scintillator signals.

Conversely, the high-energy detection layer 82 is formed of high-energy readout circuitry 104 and a high-energy scintillator 100 that has a depth and/or composition that interacts with higher energy X-rays 106 in the emitted spectra that pass through the low-energy layer 80. The interaction between the higher-energy X-rays 106 and the high-energy scintillator 100 generates optical wavelength photons or other photons detectable by the high-energy signal readout circuitry 104 (e.g., photodiodes and associated readout circuitry) positioned to detect the photons generated by interaction of X-rays 106 and the high-energy scintillator 100. The readout circuitry 104 in turn generates electrical signals 110 indicative of the intensity of X-ray radiation interacting with the high-energy scintillator 100. As with the low-energy scintillator 88, the high-energy scintillator 100 may be subdivided in one- or two-dimensions by reflectors or septa so as to be a pixelated scintillator, with pixels of the scintillator 100 corresponding to readout elements of the readout circuitry 104. For the purpose of nomenclature and to facilitate discussion herein, the electrical signals 110 generated by X-ray interactions with the high-energy scintillator are referred to as high-energy scintillator signals. For both the low- and high-energy layers 80, 82 the respective readout circuitry is depicted in-line with the X-ray transmission and stacked scintillators 88, 100. However, it should be appreciated that aspects of the readout circuitry may also or instead be positioned to the side of the stacked arrangement or elsewhere in the stack.

As may be appreciated from this discussion, the dual-layer detector 28 separates an incident X-ray beam (i.e., a single incident X-ray spectrum) into two energy distributions based on whether the X-ray photons are stopped by the low-energy (i.e., top) scintillator 88 or the high-energy (i.e., bottom) scintillator 100. That is, from a single emission spectrum, the dual-layer detector 28 separates the spectrum into a high-energy distribution corresponding to X-rays stopped by the high-energy scintillator 100 and a low-energy distribution corresponding to X-rays stopped by the low-energy scintillator 88 attributable to signals 96 read out from the low-energy readout circuitry 92, with respective signals 96, 110 reflecting X-ray incidence on the respective low-energy scintillator 88 and high-energy scintillator 100 . The issue of spectral overlap as discussed herein is due to the incomplete energy separation that may be present in the low-energy scintillator signal 96, which is obtained from the single, high kV X-ray exposure. In particular, for a given exposure event a portion of the high-energy X-rays 106 within the exposure spectrum will be stopped by, and generate signal in, the low-energy layer 80, leading to poor energy separation, while relatively few of the low-energy X-rays 90 will penetrate to, and generate signal in, the high-energy layer 82.

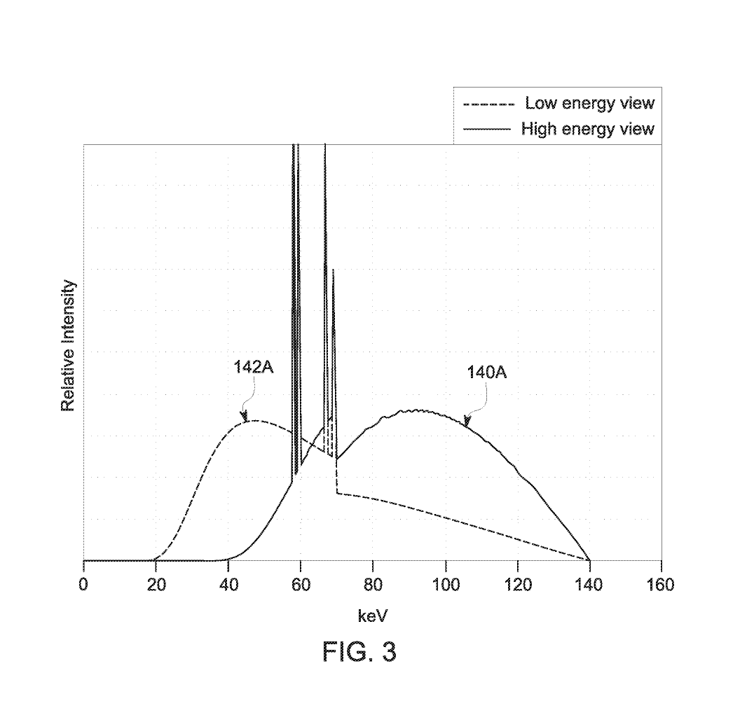

As example of the high-energy spectrum 140A and low-energy spectrum 142A that may be obtained using a dual-layer detector 28 is shown in FIG. 3. As may be observed in FIG. 3, the respective high- and low-energy spectra 142A, 140A exhibit substantial overlap across their combined range, and may overlap to some extent up to the maximum keV, as shown.

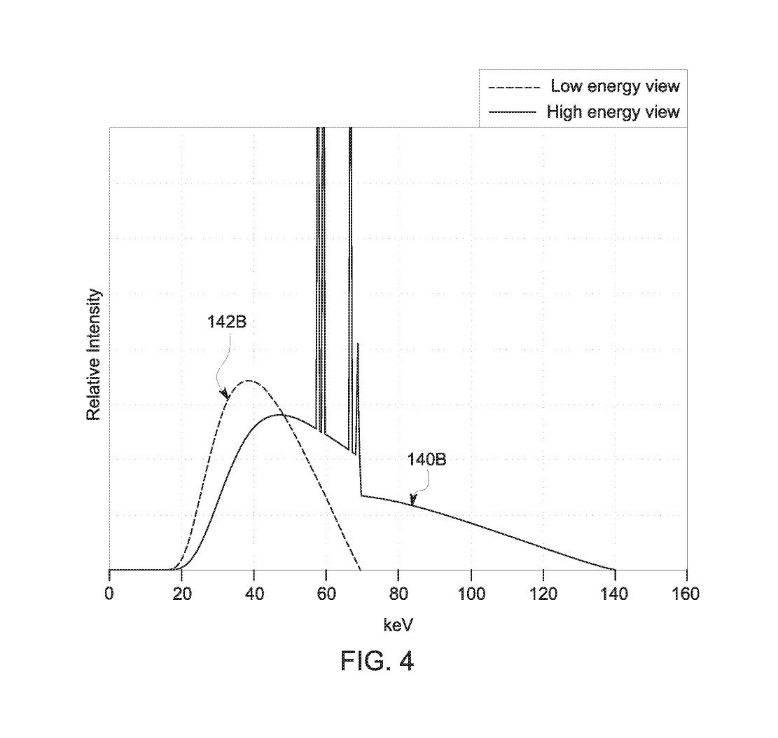

Conversely, as noted above, kV switching approaches employ a single X-ray detection mechanism or layer, but instead alternate the X-ray source emissions between a higher-energy and a lower-energy spectrum, which are separately readout at the single layer detector. Such an approach exhibits a superior low-energy spectrum 142B, as shown in FIG. 4, in that the low-energy spectrum 142B is substantially narrower and better defined with respect to the low-energy peak and exhibits little overlap with the high-energy portion of the high-energy emission spectrum 140B. However, the emitted high-energy spectrum 140B still includes low-energy X-ray photons (due to the emission being over a broad range, even when shifted toward the higher target energy), which are detected by the detector mechanism and lead to a substantial overlap of the high-energy spectrum 140B with the low-energy spectrum 142B.

In accordance with aspects of the present approach a kV switched X-ray source 12 is used in conjunction with a dual-layer detector 28. Though the present examples may relate or convey specific energy ranges or levels (such as 70 keV, 80 keV, 140 keV, 150 keV and so forth) it should be understood that all such stated energy levels are provided merely as examples and the present approach may be employed with these and other X-ray energies.

With this in mind, during a low-energy X-ray emission by the switched source 12, the emission spectrum corresponds to the narrow, well-defined low energy spectrum 142B shown in FIG. 4. As a result, little to no signal should be generated at the high-energy scintillator 100 as the emitted X-ray photons do not overlap with the higher energy region (e.g., the emitted low-energy spectrum ends at 70 keV in the depicted example). That is, the emitted low-energy X-rays are substantially all stopped at the low-scintillator 88. As a result, for this low-energy emission phase, the high-energy scintillator signal 110 should be effectively zero, allowing this signal to be discarded when read out or, if there is a trivial non-zero value of the signal 110, it may be summed with the low-scintillator signal 96 for downstream processing as it represents low-energy X-ray photons that by chance penetrated the low-energy scintillator and were stopped by the high-energy scintillator. For practical purposes, however, it may be assumed that the low-energy scintillator signal 96 represents the readout data for the low-energy X-ray emission spectrum 142B.

Conversely, during a high-energy X-ray emission by the switched source 12, the emission spectrum corresponds to a broadly polychromatic spectrum 140B that overlaps with the low-energy region of interest such as in the region below 70 keV in one example. However, due to the use of the dual-layer detector 28, the low-energy X-ray photons present in the high-energy X-ray emission spectrum 140B are stopped at the low-energy scintillator 88. Thus, by discarding the low-energy scintillator signal 96 during high-energy emissions, the high-energy scintillator signal 110 may be employed for downstream processing and will not have substantial overlap or contribution from the low-energy X-ray photons.

Thus, in one embodiment, in a low-energy X-ray emission phase of a switched source, at least the low-energy scintillator signal 96 is read out and used in downstream processing and representative of X-ray transmission at low X-ray energies. The high-energy scintillator signal 110 during this low-energy emission phase may be discarded from such downstream processing or added to the low-energy scintillator signal 96 as likely being indicative of low-energy X-rays photons that by chance reached the high-energy scintillator 100.

Conversely, in this embodiment, during the high-energy X-ray emission phase of the switched source, the low-energy scintillator signal 96 may be read out but is discarded or otherwise not used in downstream processing. The high-energy scintillator signal 110 during this high-energy emission phase, however, is retained and used in downstream processing.

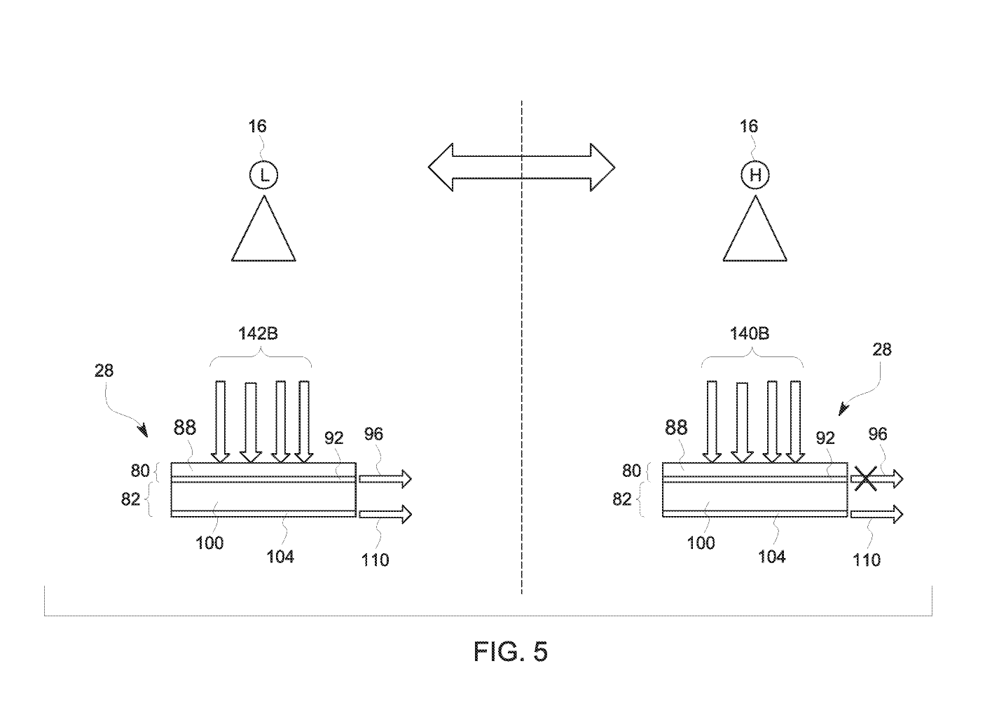

An example of this approach is illustrated pictorially in FIG. 5, where an X-ray source 16 is alternated between emitting X-ray photons in a low-energy mode (shown on the left of FIG. 5) in which the emitted X-rays exhibit a low-energy spectrum and a high-energy mode (shown on the right of FIG. 5) in which the emitted X-rays exhibit a high-energy spectrum. When the low-energy spectrum 142B is incident on the dual-layer detector 28 at least a low-energy scintillator signal 96 is acquired for subsequent processing. The high-energy scintillator signal 110 may also be acquired during the low-energy X-ray emission and will likely have a zero or near-zero value. As a result, the high-energy scintillator signal may be discarded from subsequent processing or may be added to the low-energy scintillator signal 96 for subsequent processing due to likely being attributable to some small number of low-energy X-ray photons that pass through the low-energy scintillator 88 to impact the high-energy scintillator.

Conversely, when the high-energy spectrum 140B is incident on the dual-layer detector 28, a high-energy scintillator signal 110 is acquired and used in downstream processing. The low-energy scintillator signal 96, conversely, if read out, is discarded and not used in downstream processing. Thus, and low-energy X-ray photons present in the incident high-energy spectrum 140B that interact with the low-energy scintillator 88 are not processed and improve the energy separation of the system.

While a conventional dual-layer detector 28 and dual-layer readout approach may be used in certain implementations, it should also be appreciated that variations to this approach may be employed. For example, a comparable result may be obtained using a dual-layer detector 28 having different efficiencies for low- and high-energy collection that can be controlled externally. In such an embodiment, during the high-energy acquisition, the detector 28 may be tuned to accept high energy photons only. In this manner, low energy photons would be ignored or otherwise not measured through the inherent inefficiency of the detector scheme.

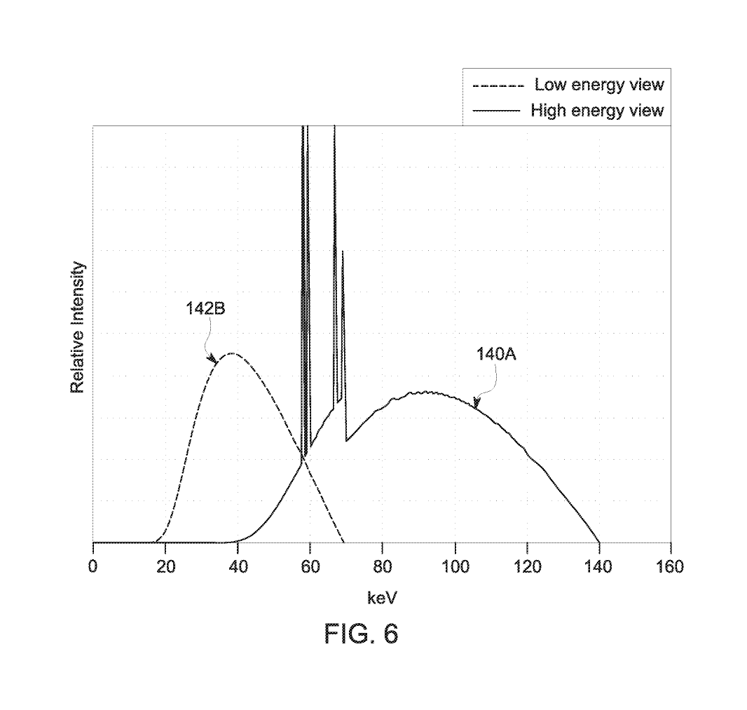

An example of observed or measured spectra in accordance with these approaches can be seen in FIG. 6. As shown in this example, the high-energy spectrum 140A and low-energy spectrum 142B are substantially better separated, with limited overlap, in comparison to either dual-layer detector or kV switched approached used in isolation.

As may be appreciated, in accordance with this approach, the effect of filtering the X-ray beam may be obtained without employing a physical or mechanical filter. With this in mind, the dual-layer detector 28 may adhere to less stringent requirements and/or use photodiodes attuned to different wavelengths than a conventional dual-layer detector since the low energy layer 80 could be shut off or otherwise not employed during the high-energy emission intervals, thus potentially allowing for less stringent electrical requirements and/or greater tolerances in timing or readout aspects. Further, the patient dose in accordance with this approach would be no greater than what would be employed in a conventional kV switching approach.

Technical effects of the invention include employing a kV switched X-ray source, such as a kV switched X-ray tube, in conjunction with a dual-layer detector. In such approaches, the dual-layer detector may be operated so as to ignore or discard signal attributable to low-energy photons generated during a high kV emission interval or view. In one such implementation, an X-ray source is alternated between emitting X-ray photons in a low-energy mode and a high-energy mode. When the low-energy spectrum is incident on the dual-layer detector at least a low-energy readout signal is acquired. When the high-energy spectrum is incident on a dual-layer detector a high-energy readout signal is acquired and no low-energy signal is acquired. In this manner, low-energy X-ray photons are separated out during the high-energy exposure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method of acquiring and processing dual-energy X-ray transmission data, comprising:
alternately emitting from an X-ray source a first X-ray beam having a first keV distribution and a second X-ray beam having a second keV distribution different than the first keV distribution;
in response to each emitted first X-ray beam, reading out at least a low-energy scintillator signal from a first layer of a dual-layer detector;
in response to each emitted second X-ray beam, reading out at least a high-energy scintillator signal from a second layer of the dual layer detector; and
processing at least the low-energy scintillator signals and the high-energy scintillator signals to generate an image.

2. The method of claim 1, wherein processing at least the low-energy scintillator signals and the high-energy scintillator signals to generate an image further comprises acquiring an additional high-energy scintillator signal for each emitted first X-ray beam and combining the additional high-energy scintillator signal with the low energy scintillator signal to generate an aggregate low-energy signal used to generate the image.

3. The method of claim 1, wherein the first keV distribution is a lower energy spectrum than the second keV distribution.

4. The method of claim 1, wherein the image is a tissue-type or material decomposition image.

5. The method of claim 1, wherein the X-ray source is a fast kV switched X-ray source.

6. The method of claim 1, further comprising:
rotating the X-ray source and dual-layer detector about an imaged volume during operation.

7. The method of claim 1, wherein the first layer of the dual-layer detector comprises:
a first scintillator material having a first thickness; and
a first readout circuitry configured to detect photons generated by the first scintillator material and to generate the low-energy scintillator signals in response.

8. The method of claim 7, wherein the second layer of the dual-layer detector comprises:
a second scintillator material different from the first scintillator material; and
a second readout circuitry configured to detect photons generated by the second scintillator material and to generate the high-energy scintillator signals in response.

9. The method of claim 7, wherein the second layer of the dual-layer detector comprises:
a second scintillator material or the first scintillator material at a second thickness different from the first thickness; and
a second readout circuitry configured to detect photons generated by the scintillator material of the second layer and to generate the high-energy scintillator signals in response.

10. An imaging system, comprising:
an X-ray source configured to be switched during operation between a first operating voltage corresponding to a first emission spectrum and a second operating voltage corresponding to a second emission spectrum;
a dual-layer X-ray detector having a first layer and a second layer;
a data acquisition system configured to read out at least the first layer when the X-ray source is operated at the first operating voltage and to read out at least the second layer when the X-ray source is operated at the second operating voltage; and
image processing circuitry configured to generate an image using signals acquired from at least the first layer when the X-ray source is operated at the first operating voltage and using signals acquired from only the second layer when the X-ray source is operated at the second operating voltage.

11. The imaging system of claim 10, wherein the first operating voltage and the second operating voltage are in a range between about 70 kVp and about 150 kVp.

12. The imaging system of claim 10, wherein the image is a tissue-type or material decomposition image.

13. The imaging system of claim 10, wherein the first emission spectrum is a lower energy spectrum than the second emission spectrum

14. The imaging system of claim 10, wherein the X-ray source is a fast kV switched X-ray source.

15. The imaging system of claim 10, further comprising a rotational structure on which the X-ray source and dual-layer X-ray detector are mounted.

16. The imaging system of claim 10, wherein the first layer of the dual-layer X-ray detector comprises:
a first scintillator material having a first thickness; and
a first readout circuitry configured to detect photons generated by the first scintillator material and to generate low-energy signals in response.

17. The imaging system of claim 10, wherein the second layer of the dual-layer X-ray detector comprises:
a second scintillator material different from the first scintillator material; and
a second readout circuitry configured to detect photons generated by the second scintillator material and to generate high-energy signals in response.

18. The imaging system of claim 10, wherein the second layer of the dual-layer X-ray detector comprises:
a second scintillator material or the first scintillator material at a second thickness different from the first thickness; and
a second readout circuitry configured to detect photons generated by the scintillator material of the second layer and to generate high-energy signals in response.

19. A method for acquiring dual-energy X-ray data, comprising:
reading out at least a low-energy scintillator layer of a dual-layer detector to generate first signals when the dual-energy detector is irradiated by an X-ray source operated at a first operating voltage;
reading out a high-energy scintillator layer of the dual-layer detector to generate second signals when the dual-energy detector is irradiated by the X-ray source operated at a second operating voltage; and
generating a tissue-type or material decomposition image using the first signals and the second signals.

20. The method of claim 20, wherein the first operating voltage corresponds to a first X-ray emission spectrum and the second operating voltage corresponding to a second X-ray emission spectrum.
